(19) United States
(12) Patent Application Publication
(10) Pub. No.: US 2026/0096820 A1
(43) Pub. Date: Apr. 9, 2026

(54) Implantable Endobronchial Valve Apparatus and Method

(71) Applicant: Bard Peripheral Vascular, Inc., Franklin Lakes, NJ (US)

(72) Inventors: Andrzej J. Chanduszko, Chandler, AZ (US); Alexander G. Lastovich, Gilbert, AZ (US); Chad Modra, Phoenix, AZ (US)

(21) Appl. No.: 18/906,057

(22) Filed: Oct. 3, 2024

(51) Int. Cl. A61B 17/12; A61F 2/04

(52) U.S. Cl. CPC A61B 17/12104; A61F 2002/043

(57) ABSTRACT

An endobronchial valve designed for placement in an airway to modify lung volume to a portion of the lung. The valve comprises a frame with a collapsible and expandable configuration, and a valve member made of a flexible material attached to the frame. The valve member includes a distal valve member with a conical shape and nested within a proximal valve member with a frusto-conical shape. The distal valve member features a cavity communicating with an opening at the base, while the proximal valve member has openings of varying diameters. The base of the distal valve member is positioned through the opening of the proximal valve member, allowing for effective modification of lung volume. The valve systems further include a swellable material for changing a fluid flow rate over time to gradually modify lung volume and mitigate complications such as pneumothorax.

### BACKGROUND

Existing devices for modifying lung volume typically involve invasive procedures such as surgery, or bronchoscopy to implant valves in the airways. Such techniques are often used to manage conditions such as emphysema or chronic obstructive pulmonary disease (COPD). Emphysema is one of a group of lung conditions called chronic obstructive pulmonary disease (COPD), which is a disease that causes breathing difficulties and serious complications if untreated. Patients with more serious emphysema who have tried medications and lifestyle changes without success may need lung volume reduction surgery. While effective, the surgery is still invasive and leads to undesirable complications such as major pulmonary and/or cardiovascular complications.

Less invasive treatment options have been under development, including bronchoscopic lung volume reduction using endobronchial valves. The main function of endobronchial valves is to significantly reduce flow of air into the diseased portion of the lung while allowing air to escape during exhalation. These valves should also allow for drainage of airway secretions. However, these devices are often difficult to reposition, or retrieve, and so may not provide the flexibility required. Additionally, the placement of these valves may result is sudden changes in lung volume that can result in additional complications to the patient such as pneumothorax, commonly termed “collapsed lung.” Placement of the endobronchial valve cause a sudden reduction in usage of the diseased portion of the lung. The diseased portion of the lung rapidly reduces in size creating a void within the chest cavity. Healthy lung tissue expands to fill this void, but doing so rapidly leads to trauma.

What is needed therefore is a system and method of gradually reducing lung volume over a period of time to mitigate sudden changes in lung volume that may lead to complications such as pneumothorax. Disclosed herein are implantable endobronchial valve systems and methods directed to address the foregoing.

### SUMMARY

In some aspects, the techniques described herein relate to a valve for placement in an airway to modify lung volume including, a frame having a body extending along a longitudinal axis between a proximal end and a distal end, the frame transitionable between a collapsed configuration and an expanded configuration, and a valve member formed of a flexible material and coupled to the frame including, a distal valve member having a conical shape extending between an apex disposed distally and a base disposed proximally and defining a cavity communicating with an opening at the base, the base defining a first diameter, and a proximal valve member defining a frusto-conical shape extending between a distal end and a base, the distal end having an opening and defining a second diameter (D2) less than the first diameter (D1), the base having an opening and defining a third diameter (D3) larger than the first diameter (D1), the base of the distal valve member disposed through the distal end opening of the proximal valve member.

In some aspects, the techniques described herein relate to a valve, wherein one or both of the base of the distal valve member and the base of the proximal valve member is configured to deflect radially inwards to provide proximal fluid flow, and where the base of the distal valve member impinges on the distal end opening of the proximal valve member to provide a reduced fluid flow.

In some aspects, the techniques described herein relate to a valve, wherein the base of the distal valve member includes a plurality of ridges and troughs disposed about a circumference of the base and configured to provide one or more gaps for distal fluid flow when the distal valve member base engages the proximal valve member distal opening.

In some aspects, the techniques described herein relate to a valve, wherein one or both of the distal valve member and the proximal valve member includes a coating having a swellable material configured to expand over a predetermined time frame and occlude the one or more gaps to reduce or prevent distal fluid flow.

In some aspects, the techniques described herein relate to a valve, wherein one or both of the distal valve member and the proximal valve member includes a plurality of apertures to allow fluid flow therethrough in both the proximal direction and the distal direction.

In some aspects, the techniques described herein relate to a valve, wherein one or both of the distal valve member and the proximal valve member includes a coating having a swellable material configured to expand over a predetermined time frame and occlude the plurality of apertures to reduce or prevent distal fluid flow.

In some aspects, the techniques described herein relate to a valve, wherein the frame body defines cylindrical shape having a diameter in the expanded configuration that is equal to, or larger than a diameter of a target location, the frame body includes one or more anchors that protrude from the frame body in the expanded configuration to engage a wall of the target location to prevent migration of the valve.

In some aspects, the techniques described herein relate to a valve, wherein the frame body includes a lattice structure of a plurality of struts, the frame further includes a distal section including two or more struts extending from the frame body to the distal end of the frame to form a conical shape and configured to retain the valve therein.

In some aspects, the techniques described herein relate to a valve, wherein the frame further includes a proximal section including two or more struts extending from the frame body to the proximal end of the frame, the proximal end includes an engagement feature.

In some aspects, the techniques described herein relate to a valve, further including one or more flexible members coupling a perimeter of the base of the proximal valve member to the frame and configured to allow the proximal valve member to move along the longitudinal axis relative to the frame between an open and a closed position.

In some aspects, the techniques described herein relate to a valve, further including a biasing member coupling the distal end of the proximal valve member to the distal end of the frame and configured to allow the proximal valve member to move along the longitudinal axis relative to the frame between an open and a closed position.

In some aspects, the techniques described herein relate to a valve, wherein a perimeter of the base of the proximal valve member is coupled to the proximal end of the frame body and further includes a proximal section coupled to the proximal end of the frame body, the proximal section including a plurality of struts extending from the frame body to a proximal end of the frame, the proximal end including an engagement feature.

In some aspects, the techniques described herein relate to a valve, wherein one or both of the distal valve member and the proximal valve member includes a relatively rigid section disposed distally and a relatively flexible section disposed proximally.

In some aspects, the techniques described herein relate to a method of placing a valve at a target location for modifying a lung volume including, advancing the valve in a collapsed configuration and disposed within a delivery catheter to the target location, the valve including a frame and a valve member, transitioning the valve to an expanded configuration for an anchor of the frame engages a wall of the target location, and transitioning the valve member to an expanded configuration to reduce distal fluid flow across the valve.

In some aspects, the techniques described herein relate to a method, further including, coupling a distal end of a stylet, disposed within a catheter lumen, with an engagement feature at a proximal end of the frame, withdrawing the stylet proximally into the catheter lumen, impinging a strut of a proximal section of the frame on an entrance of the catheter lumen, levering the frame radially inwards to disengage the anchor away from the wall of the target location, transitioning the frame to the collapsed configuration, and withdrawing the valve into catheter lumen.

In some aspects, the techniques described herein relate to a method, wherein the valve member transitions between an open position to allow proximal fluid flow, and a closed position to modify distal airflow, an outer surface of a base of a distal valve member impinges on an inner surface of a distal opening of a proximal valve member.

In some aspects, the techniques described herein relate to a method, wherein the valve member in the open position further includes deflecting the base of the distal valve member radially inward to provide a gap between the base of the distal valve member and the opening of the proximal valve member.

In some aspects, the techniques described herein relate to a method, wherein the valve member in the open position includes sliding the proximal valve member relative to the frame, the valve further including a flexible member coupled to a base of the proximal valve member, or a biasing member coupled to an apex of the valve, biasing the proximal valve member to the closed position.

In some aspects, the techniques described herein relate to a valve system for modifying a lung volume including, a valve member defining a conical shape extending proximally from an apex to a base, and defining a cavity communicating with an opening at the base, a frame including a plurality of struts extending distally from a proximal end of the frame to the base of the valve member, and two or more arms extending from the proximal end of the frame to engage a wall of a target location, and an engagement feature coupled to the proximal end of the frame.

In some aspects, the techniques described herein relate to a valve system, wherein the arm further includes one or more anchors configured to engage the wall and mitigate movement of the valve system in one or both of a proximal direction and a distal direction.

In some aspects, the techniques described herein relate to a valve system, further including a delivery catheter and a stylet disposed within a lumen of the delivery catheter, the stylet configured to engage the engagement feature and withdraw frame into the catheter lumen, the two or more arms configured to engage the catheter to lever the anchors radially inwards away from the wall of the target location.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of an endobronchial valve system including a frame and a valve member, in accordance with embodiments disclosed herein.

FIG. 1B shows a perspective view of a valve member of the endobronchial valve system of FIG. 1A, in accordance with embodiments disclosed herein.

FIGS. 2A-2C show lateral cross-sectional views of the valve member of FIG. 1B, in accordance with embodiments disclosed herein.

FIGS. 3A-3B show longitudinal cross-sectional views of an endobronchial valve system, in accordance with embodiments disclosed herein.

FIG. 4A shows a perspective view of an endobronchial valve system including a frame and a valve member, in accordance with embodiments disclosed herein.

FIG. 4B shows a longitudinal cross-sectional view of the endobronchial valve system of FIG. 4A during exhalation, in accordance with embodiments disclosed herein.

FIG. 4C shows a longitudinal cross-sectional view of the endobronchial valve system of FIG. 4A during inhalation, in accordance with embodiments disclosed herein.

FIG. 5A shows a perspective view of an endobronchial valve system including a frame and a valve member in an expanded configuration, in accordance with embodiments disclosed herein.

FIG. 5B shows a perspective view of an endobronchial valve system including a frame and a valve member in a collapsed configuration, in accordance with embodiments disclosed herein.

FIG. 6A shows a perspective view of an endobronchial valve system including a frame and a valve member in an expanded configuration, in accordance with embodiments disclosed herein.

FIG. 6B shows a perspective view of the endobronchial valve system of FIG. 6A during inhalation, in accordance with embodiments disclosed herein.

FIG. 6C shows a perspective view of the endobronchial valve system of FIG. 6A during exhalation, in accordance with embodiments disclosed herein.

FIGS. 7A-7B show perspective views of endobronchial valves including a retrieval frame system, in accordance with embodiments disclosed herein.

FIG. 8A shows a perspective view of an endobronchial valve system including a frame and a valve member during inhalation, in accordance with embodiments disclosed herein.

FIG. 8B shows a perspective view of an endobronchial valve system including a frame and a valve member during exhalation, in accordance with embodiments disclosed herein.

FIG. 8C shows a perspective view of the endobronchial valve system of FIG. 8A during placement at a target location, in accordance with embodiments disclosed herein.

FIG. 8D shows a perspective view of the endobronchial valve system of FIG. 8A once placed at a target location, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.”An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near, or relatively nearer to, a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near, or relatively nearer to, a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

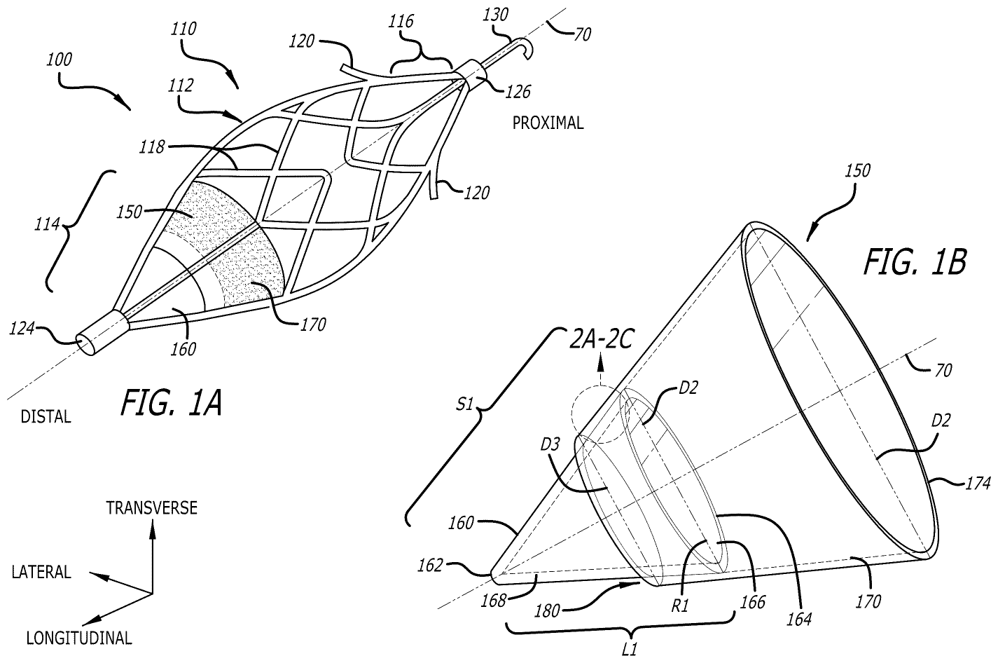

To assist in the description of embodiments described herein, as shown in FIG. 1A, a longitudinal axis extends substantially parallel to an axial length of the valve system 100. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

Embodiments described herein are directed to endobronchial valve systems and methods that include improved retrievability and repositioning, reduced migration once placed, and mitigate the occurrence of pneumothorax and similar complications. Embodiments disclosed herein include expandable cages or frames to facilitate placement, removal and repositioning of the endobronchial valve system, anchors to mitigate migration of the endobronchial valve once placed and provide incomplete or delayed closure of the airway so as to provide a gradual reduction in lung volume over time, mitigating complications from sudden reduction in lung volume. As will be appreciated, embodiments disclosed herein are exemplary and the specific size, shape, and geometry can be modified and remain within the scope of this invention.

FIGS. 1A-1B show an embodiment of an endobronchial valve system (“system”) 100 generally including a frame 110 and a valve 150. FIG. 1B shows the valve 150 separate from the system 100 for ease of illustration. In an embodiment, the frame 110 extends between a proximal end 126 and a distal end 124 and includes a frame body 112 disposed therebetween defining a substantially cylindrical cross-sectional shape. In an embodiment, the frame 110 further includes a valve section 114 disposed distally and a retrieval section 116 disposed proximally.

In an embodiment, the system 100 is configured to be placed within a bronchial airway with the distal end 124 placed towards the bronchioles and further from the trachea, and the proximal end 126 placed towards the trachea and further from the bronchioles. As such during inhalation, distal air flow will be from the proximal end 126 to the distal end 124 of the system 100, and during exhalation, proximal air flow will be from the distal end 124 to the proximal end 126 of the system 100.

In an embodiment, one or more portion of the frame 110, e.g., the frame body 112 can be formed as a lattice or mesh structure. The frame 110 is configured to transition between a collapsed configuration, defining a first diameter, and an expanded configuration, defining a second diameter, larger than the first diameter. In an embodiment, the second diameter can be between 4 mm-10 mm, however, greater or lesser diameters are also contemplated. In the collapsed configuration, the system 100 as a whole can be placed within a placement catheter 90 and advanced through the trachea and bronchi to a target location within the lung. The system 100 can then be advanced from the placement catheter 90 and can transition to the expanded configuration at the target location. In an embodiment, the system 100 is biased towards the expanded configuration and is elastically deformed to the collapsed configuration and maintained in the collapsed configuration by the placement catheter 90.

In an embodiment, the frame 110 can be formed of a plastic, polymer, metal, alloy, composite, shape-memory material, super-elastic material, a nickel titanium alloy, Nitinol, stainless steel, cobalt chromium alloys, or similar suitable material. In an embodiment, the frame body 112 is formed of a shape-memory alloy, or similar material and transitions from the collapsed configuration to the expanded configuration by a change in temperature, or similar trigger. It will be appreciated that other physiological triggers are also contemplated such as a contact with fluid, change in pH, or the like.

In an embodiment, the frame body 112 is formed of a lattice or mesh structure having a plurality of struts 118 coupled together. As shown the plurality of struts 118 can form a diamond shaped lattice, however, other configurations, shapes, sizes, lengths, thicknesses of struts 118 are also contemplated. The plurality of struts 118 can be formed integrally to form the lattice of the frame body 112. In an embodiment, the plurality of struts 118 can be coupled together to form the frame body 112 using adhesive, bonding, welding, ultrasonic welding, RF welding, or similar suitable techniques. In an embodiment, the plurality of struts 118 can be elastically deformable to allow the frame 110 to transition between the collapsed configuration and the expanded configuration. In an embodiment, the joints between the plurality of struts 118 can be elastically deformable to allow the frame 110 to transition between the collapsed configuration and the expanded configuration. In an embodiment, a spacing between the plurality of struts 118 of the frame 110 can be of sufficient size so as to allow a fluid (gas, liquid, or combination thereof) to flow through the frame 110 with little or no resistance.

In an embodiment, the frame 110 further includes one or more anchors 120. The anchors 120 can include a barb or spike extending from the frame body 112 in an expanded configuration and at an angle to a central longitudinal axis 70 of the system 100. In an embodiment, the anchors 120 are formed of the same material, or of a different material, from that of the frame 110. In the expanded configuration the anchors 120 extend from an outer circumference of the frame body 112 to engage a wall of the bronchus at the target location and prevent the system 100 from migrating once placed. In an embodiment, with the frame 110 in the collapsed configuration, the anchors 120 are aligned substantially parallel with the longitudinal axis of the system 100, and/or the ends of the anchors 120 are disposed within the outer circumference of the frame body 112, so as to mitigate engagement with inner surface of the placement catheter 90 and/or the wall of the bronchus prior to transitioning to the expanded configuration.

In an embodiment, the frame 110 includes a first set of one or more anchors 120 that are angled towards the distal end 124 in the expanded configuration and a second set of one or more anchors 120 that are angled towards the proximal end 126 in the expanded configuration. The first set of one or more anchors 120 are configured to engage a wall surface of the bronchus at the target location and mitigate movement of the system 100 in a distal direction. The second set of one or more anchors 120 are configured to engage a wall surface of the bronchus at the target location and mitigate movement of the system 100 in a proximal direction. Transitioning the frame body 112 from the expanded configuration to the collapsed configuration can withdraw the one or more anchors 120 from the wall surface of the target location and allow the system 100 to be repositioned or removed.

In an embodiment, a proximal end of the frame 110 includes an engagement feature 130, such as a hook, loop, or similar structure configuration to engage a distal end of a stylet 80, snare, or similar retrieval device. The distal end of the stylet 80 is configured to engage the hook 130 to withdraw the system 100 proximally, retain the system 100 as the delivery catheter 90 is urged distally relative to the system 100, and/or transition the system 100 between an expanded configuration and a collapsed configuration, or combinations thereof.

In an embodiment, the retrieval section 116 includes one or more struts 118 extending from the proximal end 126, disposed centrally, to a proximal edge of the frame body 112. The one or more struts 118 of the retrieval section 116 are configured to transition between a collapsed configuration and an expanded configuration. In the collapsed configuration the one or more struts 118 are aligned substantially parallel to the central longitudinal axis 70, and in the expanded configuration the one or more struts 118 are angled relative to the central longitudinal axis 70.

In an embodiment, the retrieval section 116 is configured to provide mechanical advantage to lever the anchors 120 from the inner surface of the target location and facilitate removal of the frame 110 from the target location. For example, where the stylet 80 engages the engagement feature 130 and withdraws the frame 110 into the delivery catheter lumen 92, the struts 118 of the retrieval section 116 engage the distal end of the delivery catheter 90 and urge the struts 118 of the retrieval section 116 radially inwards. This in turn urges the proximal end of the frame body 112 radially inwards, disengaging the anchors 120 from the wall of the target location prior to withdrawing the system 100 into the delivery catheter, reducing damage to the walls of the bronchus.

In an embodiment, a valve section 114 of the frame includes one or more struts 118 extending longitudinally and defining a conical shape extending from the distal end of the frame body 112 to a distal end 124 of the frame 110. The valve section 114 can support a valve 150 disposed within the valve section 114. The valve 150 includes a distal valve member 160 and a proximal valve member 170. FIG. 1B shows the valve 150 including the distal valve member 160 and the proximal valve member 170 shown in wire frame outline and with the frame 110 removed for ease of illustration.

In an embodiment, one or both of the distal valve member 160 and the proximal valve member 170 are formed of a flexible membrane material including a plastic, polymer, elastomer, rubber, silicone rubber, polytetrafluoroethylene (PTFE), expanded polytetrafluoroethylene (ePTFE), polyurethane (PU), a gas impermeable material, combinations thereof, or similar suitable material. In an embodiment, the distal valve member 160 and the proximal valve member 170 are formed of the same material and/or display the same mechanical properties. In an embodiment, the distal valve member 160 and the proximal valve member 170 are formed of different materials and/or display different mechanical properties, e.g., flexibility, elasticity, malleability, tensile strength, etc. The distal valve member 160 defines a conical shape having an outer surface 168 that matches an inner surface of the valve section 114 of the frame 110. Worded differently, the radius (R1) from the central longitudinal axis 70, a longitudinal length (L1) along the central longitudinal axis 70, and a slant height (S1) along the distal valve member outer surface 168 matches that of an inner surface of a conical shape of the valve section 114. The distal valve member 160 further includes an inner surface 166 that defines a conical shaped cavity extending from the apex 162, disposed distally, to the base 164, disposed proximally.

In an embodiment, the proximal valve member 170 defines a frusto-conical shape extending between a distal end 172 to a base 174. The base 174 having a diameter (D2) that is greater than the diameter (D1) of the distal valve member base 164. A distal end 172 of the proximal valve member 170 defines an opening having a diameter (D3) that is less than the diameter (D1) of the distal valve member base 164. A proximal valve member base 174 defines a proximal opening such that the proximal valve member 170 defines a tapered lumen extending therethrough.

As shown in FIG. 1B a proximal end of the distal valve member 160 extends through the distal opening of the proximal valve member 170 such that the outer surface of the proximal end of the distal valve member 160 engages an inner surface of the distal opening of the proximal valve member 170. When the valve 150 is seated within the valve section 114 of the frame, and distal fluid flow will urge the distal valve member 160 distally and engage the inner surface of the proximal valve member 170. The valve 150 is then retained in place by the struts of the valve section 114 of the frame.

In an embodiment, the base radius (R2) of the proximal valve member can be equal to half the diameter of the frame in the expanded configuration. Where the diameter of the frame in the expanded configuration is equal to the diameter of the lumen of the target location, the distal valve member 160 and the proximal valve member 170 of the valve 150 can co-operate to reduce or even prevent distal fluid flow past the system 100 during inhalation.

In an embodiment, the diameter of the base of the proximal valve member 170 can be less than the diameter of the frame body 112 in the expanded configuration. As such, during inhalation some fluid flow can pass between the outer circumference of the proximal valve member base 174 and the inner wall of the target location to allow a reduced air flow in a distal direction.

During exhalation, proximal fluid flow can urge past the distal valve member 160 and the proximal valve member 170 of the valve 150 allowing fluid flow past the system 100 in a proximal direction. In an embodiment, one or both of the distal valve member 160 and the proximal valve member 170 are coupled to the struts 118 of the valve section 114 of the frame 110 to retain the distal valve member 160 and/or proximal valve member 170 in the valve section 114 during both inhalation and exhalation. In an embodiment, an apex 162 of the distal valve member 160 is coupled to the distal end 124 of the frame 110. In an embodiment, a portion of the perimeter of the distal valve member base 164 is coupled to the struts 118 of the frame 110. In an embodiment, a portion of the outer circumference of the proximal valve member base 174, e.g., disposed between the struts, can deflect inwards during proximal fluid flow to allow fluid flow to pass by the proximal valve member 170. In an embodiment, a portion of the perimeter of the proximal valve member base 174 is coupled to the frame 110 to prevent the portion from deflecting inwards during exhalation. For example, a quarter, half, or three-quarters of the perimeter can be coupled to the frame 110 allowing the remaining perimeter distance that is uncoupled to deflect inwards during exhalation. As will be appreciated these perimeter distances are exemplary and greater or lesser perimeter distances are contemplated to fall within the scope of the present invention. As such the system 100 can be modified to allow differing amounts of fluid flow past the proximal valve member 170 during exhalation.

In an embodiment, the distal valve member 160 can be configured to move along a longitudinal axis in a proximal direction during exhalation to allow an annular opening to be formed between the distal valve member outer surface 168 and the distal opening of the proximal valve member 170. In an embodiment, a portion of the outer perimeter of the distal valve member base 164 can deflect inwards during proximal fluid flow to allow fluid flow to pass between the distal valve member 160 and the distal opening of the proximal valve member 170. Advantageously, different combinations of movement and deflection of the distal valve member 160 and the proximal valve member can allow for different rates for fluid flow during inhalation and exhalation to suit the needs of the patient and/or the situation.

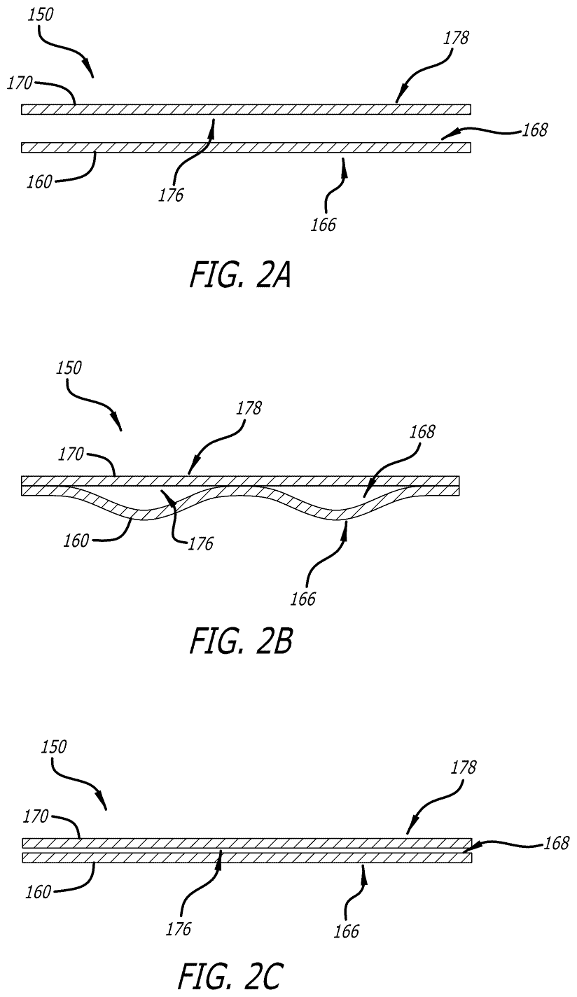

FIGS. 2A-2C show a lateral cross-section view showing the engagement between the distal valve member 160 and the proximal valve member 170. FIG. 2A shows the valve 150 in an open position during exhalation and defining a gap, or annular opening disposed between the distal valve member outer surface 168 and the proximal valve member inner surface 176 to allow proximal fluid flow. Further, as shown in FIG. 2C, during inhalation the valve 150 is in a closed position where the distal valve member outer surface 168 engages the proximal valve member inner surface 176 creating a seal and preventing any fluid flow therebetween.

In an embodiment, as shown in FIG. 2B, during inhalation, the distal valve member 160 engages the proximal valve member inner surface 176 in an incomplete seal providing gaps between the distal valve member outer surface 168 and the proximal valve member inner surface 176, providing a reduced fluid flow during inhalation. In an embodiment, the outer circumference of the distal valve member base 164 is formed having an undulating or wave-like shape. As such, where the peaks of the wave-like shaped base 164 engage the proximal valve member inner surface 176, the troughs define gaps between the distal valve member outer surface 168 and the proximal valve member inner surface 176. In an embodiment, the outer circumference of the distal valve member base 164 is formed having a regular, circular shape. The distal valve member 160 can be urged distally through the distal opening of the proximal valve member 170 until the outer edge of the distal valve member base 164 collapses forming a regular or irregular undulating perimeter shape, providing one or more gaps, as shown in FIG. 2B.

In an embodiment, a surface of one or both of the distal valve member 160 and the proximal valve member 170 includes one or more apertures disposed therein. The one or more apertures can allow for fluid flow in both the proximal and distal directions. The number, size, position, and configuration of the one or more apertures can be modified to allow for differing rates of fluid flow in both the proximal and distal directions. The one or more apertures can be formed as perforations, laser-cut holes, or the like. In an embodiment, one or both of the distal valve member 160 and the proximal valve member 170 includes a swellable material or coating disposed thereon and configured to swell over a predetermined period of time. Exemplary swellable materials can include hydrophilic coatings, hydrogels, or the like, that swell in response to the moisture within the fluid flow of the target location. The swellable material can be configured to swell over time to occlude the one or more apertures. The swellable material can be configured to gradually occlude the apertures concurrently or can expand over a surface to valve 150 occlude each aperture consecutively. Advantageously, the swellable material can provide a gradual reduction in fluid flow through the one or more apertures over time to gradually reduce lung volume.

In an exemplary method of use, an endobronchial valve system 100 is provided as described herein, for providing reduced lung volume. A target location within the bronchi is identified that provides fluid flow to a portion of the lung. The system 100 in the collapsed configuration, is placed within a delivery catheter 90 and advanced to the target location. In the collapsed configuration the frame 110 provides a reduced outer diameter and the valve 150 is folded to also provide a reduced outer diameter. At the target location the system 100 is advanced from the delivery catheter 90, optionally by urging a stylet 80 coupled to the distal end 124 in a distal direction. The distal end of the stylet 80 is releasably engaged with the system 100 at the engagement feature 130.

Once advanced from the delivery catheter 90, the system 100 can be transitioned to the expanded configuration, as described herein. In an embodiment, the system 100 is biased towards the expanded configuration and is retained in the collapsed configuration by the delivery catheter 90. In an embodiment, the system 100 is biased towards the collapsed configuration and is urged to the expanded configuration by one or both of the delivery catheter 90 and the stylet 80. In an embodiment, the frame 110 is formed of a shape memory material that transitions between the expanded configuration and the collapsed configuration in response to a trigger, e.g. a change in temperature trigger.

As the system 100 transitions to the expanded configuration, one or more anchors 120 engage a wall of the target location to prevent migration of the system 100 in either a proximal or a distal direction. As the frame 110 expands, the valve 150 can unfold to the conical shape as shown in FIG. 1B. Either the valve 150 is biased towards the unfolded, expanded configuration, and/or a portion of the valve 150 is attached to the frame 110 and is pulled to the unfolded configuration by the expansion of the frame 110. The stylet 80 is disengaged and the stylet 80 and delivery catheter 90 is withdrawn.

During exhalation, fluid flow can pass over or through the valve system 100 in a relatively uninhibited manner. The distal valve member 160 can move along a longitudinal axis in a proximal direction relative to the proximal valve member 170 to form an opening between the distal valve member outer surface 168 and the proximal valve member distal opening 180 (FIG. 2A). Alternatively, or in addition, fluid can flow pass the outer edge of the proximal valve member base 174. The diameter of the proximal valve member base 174 can be smaller than the diameter of the frame 110 and/or the diameter of the lumen of the target location to provide an annular opening in between. In an embodiment, the outer edge of the proximal valve member base 174 is configured to collapse inwards to provide an opening and allow fluid flow to pass the outer edge of the proximal valve member base 174. In an embodiment, the outer edge of the distal valve member base 164 is configured to collapse inwards to provide an opening and allow proximal fluid flow to pass through the valve system 100. One or more combinations of opening or collapsing inwards can provide relatively low fluid flow resistance during exhalation allowing air and secretions to be expelled from the target portion of the lung.

During inhalation, the valve 150 can expand to the unfolded configuration, as shown in the FIG. 1B, to reduce or inhibit fluid flow in a distal direction. The valve 150 can engage the inner surface of the struts 118 of the valve section 114 to support the valve 150. Further, the outer surface of the distal valve member 160 can form a seal with the inner surface of the proximal valve member 170 to form a complete seal as shown in FIG. 2C.

In an embodiment, the diameter of the proximal valve member base 174 is smaller than the diameter of the frame 110 and/or the diameter of the lumen of the target location to allow distal fluid flow to pass the outer edge of the proximal valve member base 174 in the distal direction. In an embodiment, the outer surface of the distal valve member 160 forms an incomplete seal (e.g., FIG. 2B) with the inner surface of the proximal valve member 170 to provide a reduced fluid flow in a distal direction, as described herein. In an embodiment, a distal fluid flow urges the distal valve member 160 through the proximal valve member distal opening 180 and cause the distal valve member base 164 to collapse or buckle, forming gaps through which distal fluid flow can pass, as shown in FIG. 2B.

Advantageously, the system 100 allows for minimal or no resistance to proximal fluid flow during exhalation. Further, the system 100 prevents fluid flow in a distal direction, during inhalation, to reduce airflow to a target location within the lung. In an embodiment, the system 100 provides a reduced fluid flow in a distal direction, during inhalation. This allows for a reduced airflow to a target location within the lung to allow for a gradual reduction in lung volume usage of the target portion of the lung, mitigating complications from a sudden reduction in lung volume.

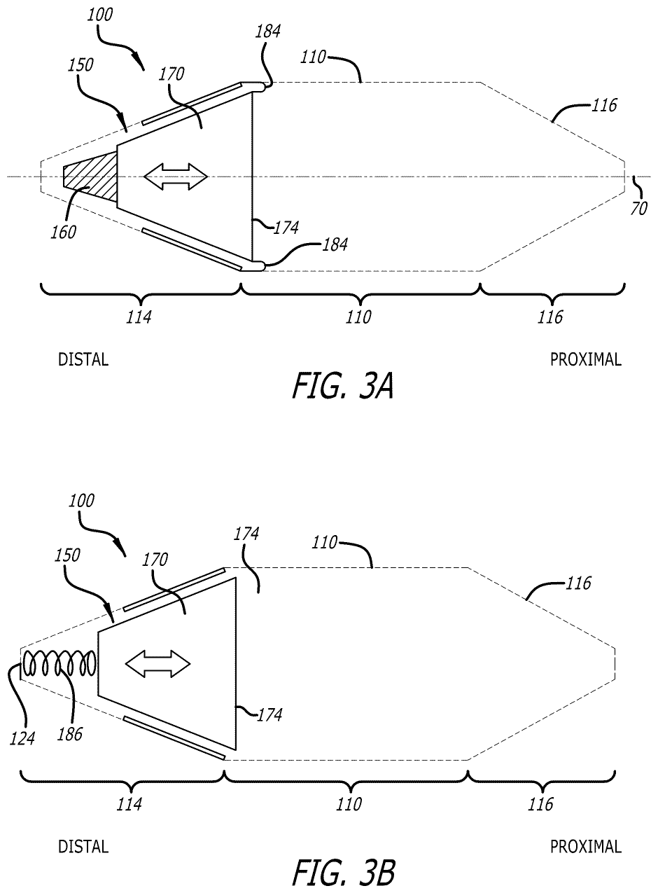

FIGS. 3A-3B shows an embodiment of the valve system 100 including a proximal valve member 170 configured to move along the longitudinal axis 70. FIG. 3A shows a longitudinal cross-sectional view of the system 100 including a frame 110 and a valve 150 disposed in the valve section 114 of the frame 110. An outer perimeter of the proximal valve member base 174 is coupled to the inner surface of the frame 110 by one or more flexible members 184 disposed about the circumference of the proximal valve member base 174. The one or more flexible members 184 couple the proximal valve member 170 to the frame 110 while still allowing the proximal valve member 170 to move along the longitudinal axis between an open position, disposed proximally, and a closed position disposed distally. In the open position, the proximal valve member 170 allows increased fluid flow between the outer surface 178 and the frame 110.

FIG. 3B shows a longitudinal cross-sectional view of an embodiment of the system 100 including a frame 110 and a valve 150 disposed in the valve section 114 of the frame 110. The distal valve member 160 is not shown for ease of illustration. In an embodiment, the proximal valve member distal end 172 is coupled to the distal end 124 of the frame 110 by a biasing member 186, such as a spring or the like. The biasing member 186 allows the proximal valve member 170 to move along a longitudinal axis between an open position, disposed proximally, and a closed position disposed distally. The biasing member 186 biases the proximal valve member 170 towards the closed position, retaining the proximal valve member outer surface 178 against the inner surface of the valve section 114 of the frame 110. In the open position, the proximal valve member 170 allows increased fluid flow between the outer surface 178 and the frame 110. In an embodiment, the biasing member 186 is coupled to the apex 162 of the distal valve member 160 and is configured to allow the valve 150 as a whole, i.e., the distal valve member 160 and the proximal valve member 170 assembly, to move along a longitudinal axis between an open position and a closed position, as described herein.

As shown in FIGS. 1A-3B, the valve 150 includes two valve members arranged concentrically with the distal valve member 160 nested within the distal opening 180 of the proximal valve member 170. In an embodiment, the valve 150 includes three or more valve members each nested within the distal opening of the adjacent valve member disposed proximally, as described herein. In an embodiment, the valve 150 includes a single conical-shaped valve member disposed within the valve section 114 of the frame 110. These and other numbers and configurations of valve members are contemplated to fall within the scope of the present invention.

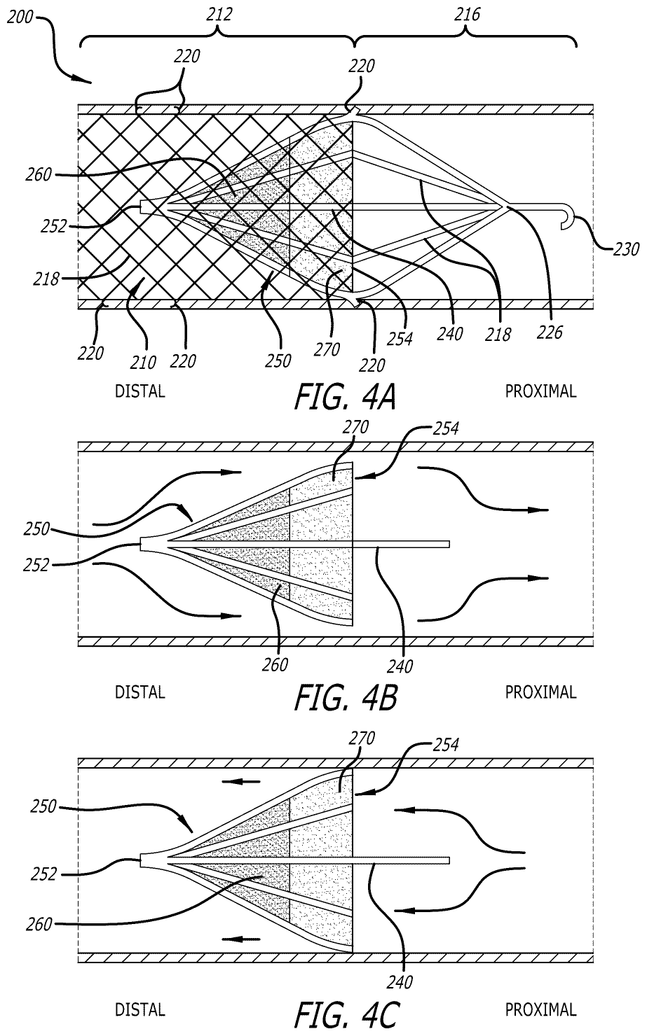

FIG. 4A shows an embodiment of an endobronchial valve system 200 generally including a frame 210 and a valve member 250 formed in a conical shape and defining a cavity. The valve system 200 is transitionable between a collapsed configuration and an expanded configuration to facilitate placement using a placement catheter 90, as described herein.

In an embodiment, the frame 210 includes a body 212 formed of a lattice or mesh structure including a plurality of struts 218, as described herein. The frame 210 can be formed of a plastic, polymer, metal, alloy, composite, shape-memory material, super-elastic material, a nickel titanium alloy, Nitinol, stainless steel, cobalt chromium alloys, or similar suitable material. The frame body 212 defines a cylindrical shape in the expanded configuration that is equal to, or greater than, the diameter of the target location. The frame 210 can include one or more anchors 220 as described herein, to engage an inner wall of the target location and mitigate proximal and/or distal migration of the valve system 200.

A retrieval section 216 of the frame 210 includes one or more struts 218 coupled to the proximal end of the frame body 212 and extending to a proximal end 226 of the frame 210. The struts 218 can be coupled to the frame body 212 using adhesive, bonding, welding, or the like. In an embodiment, the frame body 212 and the retrieval section 216 are formed integrally as a single monolithic piece. The valve system 200 further includes an engagement feature 230 extending proximally from the proximal end 226 of the frame and configured to engage a stylet 80 for positioning and/or retrieving the valve system 200, as described herein.

The valve system 200 further includes a conically shaped valve member 250. An apex 252 of the valve member 250 is disposed distally and a base 254 of the valve member 250 is coupled to a proximal end of the frame body 212 such that the valve member 250 extends distally into a lumen defined by the frame body 212. In an embodiment, a perimeter of the base 254 is coupled to the frame body 212 along an entire circumference. In an embodiment, a perimeter of the base 254 is coupled to the frame body 212 at one or more points along the circumference. In an embodiment, a perimeter of the base 254 is not coupled to the frame body 212 and the valve member 250 is retained in place by a central shaft 240 extending longitudinally from the proximal end 226 of the frame 210.

In an embodiment, the valve member 250 is formed of a flexible material such as a plastic, polymer, elastomer, rubber, silicone rubber, polytetrafluoroethylene (PTFE), expanded polytetrafluoroethylene (ePTFE), polyurethane (PU), or similar suitable material. In an embodiment, a distal portion of the valve member 250, extending from the apex 252 includes a relatively rigid section 260 of the cone. The valve member 250 further includes a relatively flexible section 270 disposed proximally and extending to the base 254.

In an embodiment, the valve member 250 can be formed of the same material with the rigid section 260 being formed of a relatively thicker material than the flexible section 270. In an embodiment, the rigid section 260 can be formed of a different material from that of the flexible section 270, that defines different mechanical properties. In an embodiment, the rigid section 260 can include one or more ribs or similar reinforcing structures, formed of the same material or a different material from that of the rigid section 260 and/or flexible section.

In an embodiment, the retrieval section 216 is configured to facilitate removal or repositioning of the valve system 200. As the engagement feature 230 is urged proximally, the struts 218, connected thereto, pull the perimeter of the base 254, and any anchors 220 disposed therewith radially inwards detaching the valve 200 from the walls of the target location with minimal damage, facilitating retrieval and repositioning of the valve 200. Alternatively, or in addition, withdrawing the engagement feature 230 back into the delivery catheter 90 urges the struts 218 to deflect radially inwards, disengaging the anchors 220 from the wall of the target location with minimal damage.

As shown in FIG. 4B, during exhalation, proximal fluid flow can pass between the outer edge of the base 254 of the valve member 250. The flexible section 270, disposed proximally, allows the base to flex radially inwards allowing the fluid flow to pass with relatively little resistance. As shown in FIG. 4C, during inhalation, distal fluid flow impinges on in inner surface of the valve member 250 causing the flexible section 270 to elastically deform radially outwards. The outer perimeter of the base 254 impinges on an inner surface of the frame 210 and/or the wall of the target location causing a partial or full occlusion of the airway and reducing or preventing distal fluid flow.

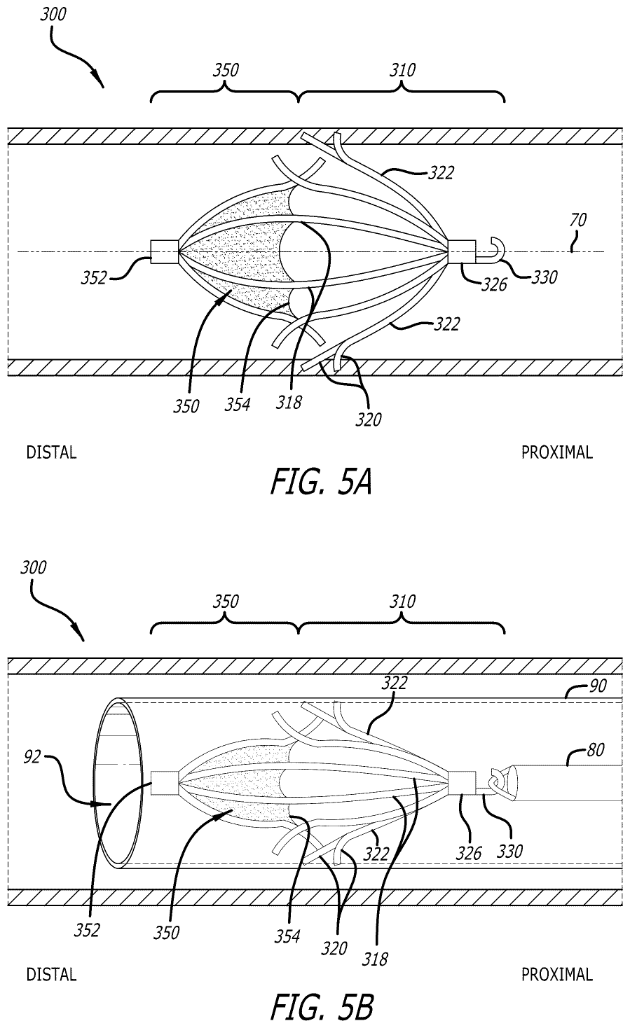

FIGS. 5A-5B show an embodiment of an endobronchial valve system 300 generally including a frame 310 having a plurality of struts 318, and a valve member 350 formed in a conical shape and defining a cavity. The frame 310 extends from a proximal end 326 aligned with a central longitudinal axis 70 and includes a plurality of struts 318 positioned radially about the central longitudinal axis 70 and extending at an angle towards an inner surface of the target location within the bronchus. A distal end of each strut of the plurality of struts 318 is coupled to a point on the perimeter of the base 354 of the valve member 350.

The plurality of struts 318 and the valve member 350 are transitionable between an expanded configuration and a collapsed configuration. In the expanded configuration the perimeter of the base 354 defines are relatively larger diameter that is equal to the diameter of the inner lumen of the target location. In the collapsed configuration the plurality of struts 318 and the valve member 350 collapse radially inwards towards to the central longitudinal axis 70.

The frame 310 further includes two or more arms 322 extending from the proximal end 326 of the frame 310 and disposed radially about the central longitudinal axis 70. The arms 322 extend at an angle outwards towards the inner surface of the target location. The distal end of each arm of the plurality of arms 322 further include one or more anchors 320 configured to engage the inner surface of the target location and prevent migration of the valve 300 in one or both of the proximal and distal direction. As shown, a first arm of the plurality of arms 322 includes a first anchor 320 extending distally and a second anchor 320 extending proximally. The first anchor 320 engages the inner surface of the target location to mitigate distal migration of the valve 300, and the second anchor 320 engages the inner surface of the target location to mitigate proximal migration of the valve 300. In an embodiment, the proximal end 326 of the frame further includes an engagement feature 330 configured to engage a stylet 80 or similar device, as described herein.

In an exemplary method of use, a valve system 300 is provided as described herein. As shown in FIG. 5B, the valve 300 is transitioned to a collapsed configuration and disposed within a delivery catheter 90. The delivery catheter 90 is advanced to the target location and the valve 300 is urged distally from the delivery catheter 90, optionally by a stylet 80 coupled to the engagement feature 330. In the collapsed configuration, the valve member 350 is in a folded configuration, the struts 318 and the arms 322 of the frame 310 are elastically deflected radially inwards towards the central longitudinal axis 70. In an embodiment, the valve 300 is biased to the expanded configuration and is retained in the collapsed configuration by the delivery catheter 90. Once the valve 300 is advanced distally of the delivery catheter 90, the struts 318 and the arms 322 return to the elastically undeformed, extended position. The distal ends of the arms 322 engage the inner surface of the target location and retain the valve in position, mitigating proximal or distal migration. The struts 318 expand radially outwards and spread the base of the valve member 350 to the expanded configuration.

During inhalation, distal fluid flow is captured within the cavity of the valve member 350 and, if not already, expands the base 354 of the valve member 350 radially outwards to mitigate or inhibit distal fluid flow. During exhalation, proximal fluid flow impinges on an outer surface of the valve member 350 causing the base 352 to fold and deflect radially inwards allowing fluid flow to pass by the valve 300 with little or no resistance. Advantageously, the arms 322 are configured to center the valve 300 along the central longitudinal axis 70 of the target location.

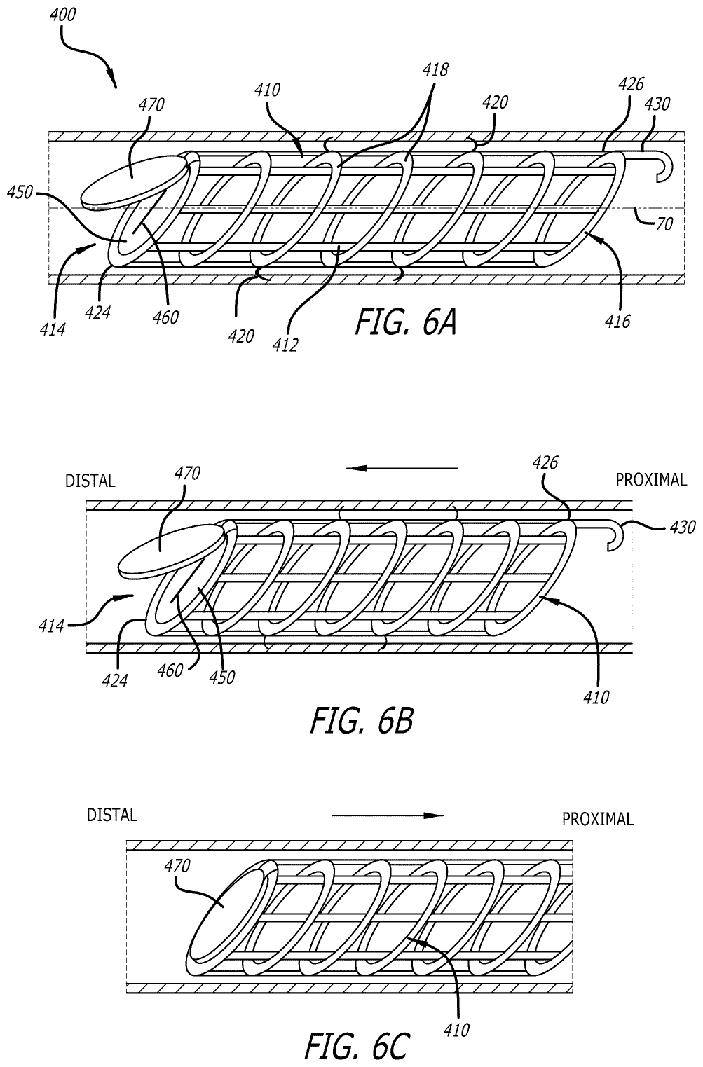

FIGS. 6A-6C show an embodiment of an endobronchial valve 400 generally including a frame 410 and a valve member 450. The frame 410 includes a frame body 412 having a mesh or lattice structure defining a substantially cylindrical shape and configured to transition between a collapsed configuration and an expanded configuration. The mesh or lattice structure can include a plurality of struts 418 and formed of a plastic, polymer, metal, alloy, composite, shape-memory material, super-elastic material, a nickel titanium alloy, Nitinol, stainless steel, cobalt chromium alloys, or similar suitable material, as described herein.

The frame 410 extends between a proximal end 426 and a distal end 424. The proximal end 426 includes a proximal opening 416 communicating with a lumen defined by the frame body 412. In an embodiment, the proximal opening 416 extends over a plane extending perpendicular, or at an angle, relative to a central longitudinal axis 70. The proximal end 426 further includes an engagement feature 430 configured to engage a stylet 80, as described herein. In an embodiment, the frame 410 further includes one or more anchors 420, as described herein, configured to engage the wall of the target location and mitigate proximal and distal migration. The distal end 424 includes a distal opening 414 communicating with the lumen defined by the frame body 412. The distal opening 414 extends over a plane extending perpendicular, or at an angle, relative to a central longitudinal axis 70.

The valve member 450 includes a membrane disposed over the distal opening 414. The valve member 450 can be formed of a flexible membrane material including a gas impermeable material, a plastic, polymer, elastomer, rubber, silicone rubber, polytetrafluoroethylene (PTFE), expanded polytetrafluoroethylene (ePTFE), polyurethane (PU), or similar suitable material. The valve member 450 further includes a slit 460 disposed therein and configured to allow a fluid flow therethrough in one or both of a proximal direction and a distal direction. The size of the slit 460 can be modified to modify a rate of fluid flow therethrough.

In an embodiment, the valve 400 further includes a flap 470, hingedly coupled to one or both of the valve member 450 and the distal opening 414. The flap 470 can be formed of the same material as the valve member 450, or from a different material, optionally displaying different mechanical properties. The flap 470 can transition between an open position (FIG. 6B) and a closed position (FIG. 6C) where the flap 470 either partially or fully covers the slit 460.

As shown in FIG. 6B, in the open position the flap 470 allows fluid flow through the slit 460 in a distal direction with little or no flow resistance over that of the slit 460. As shown in FIG. 6C, in the closed position the flap 470 restricts fluid flow through the slit 460. In an embodiment, the flap 470 covers the slit 460 entirely and prevents fluid flow through the slit 460. In an embodiment, the flap 470 partially covers the slit 460 reduces fluid flow through the slit 460 in a proximal direction. Advantageously, the size, shape and configuration of the flap 470 and/or the slit 460 can be modified to vary the rate of fluid flow through the valve 400.

As shown in FIG. 6A, the lattice of the frame body 412 is angled relative to the longitudinal axis 70 and the plane over which the valve member 450 extends can be positioned at an angle relative to the central longitudinal axis 70. For example, as shown, the valve member 450 extends over a plane extending at an angle of 45° relative to the central longitudinal axis 70. It will be appreciated that greater or lesser angles are also contemplated to fall within the scope of the present invention. Transitioning the valve 400 to the collapsed configuration, the frame folds such that the plane over which the valve member 450 extends is at a more acute angle, or even parallel with, the longitudinal axis 70. Once placed at target location, the frame 410, which is biased towards the expanded configuration, expands to a relatively less acute angle to fill the diameter of the target location. Advantageously, the same valve 400 can be configured to fit different diameter target locations since the frame body 412 can expand from the acute angle until the outer surface of the frame body 412 engage the walls of the target location. For smaller diameter target locations, the frame body 412 will be at a relative more acute angle when the frame body 412 engages the walls of the target location. For larger diameter target locations, the frame body 412 will be at a relative less acute angle when the frame body 412 engages the walls of the target location.

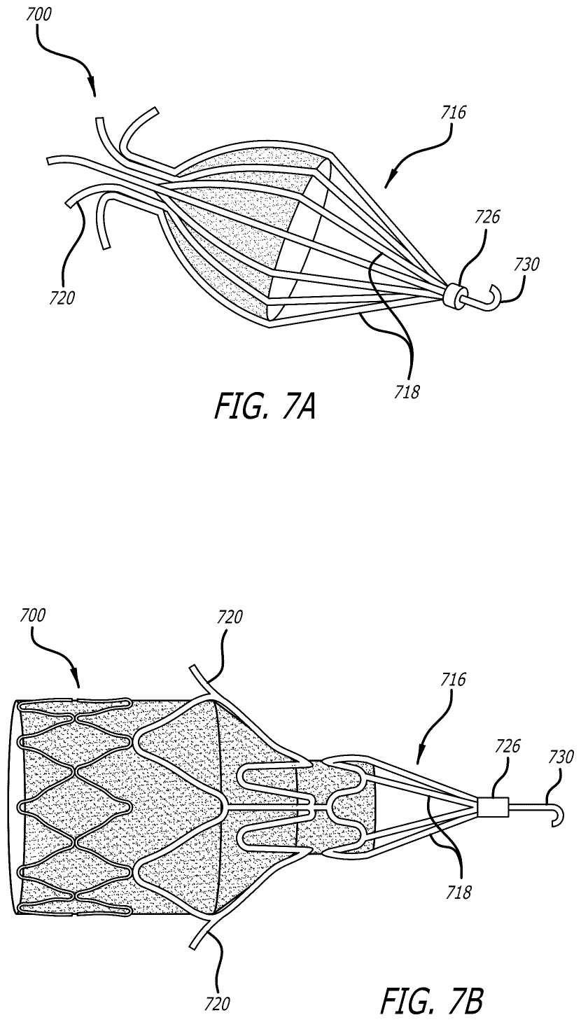

FIGS. 7A-7B show additional embodiments of bronchial valves 700 configured to be placed at a target location to modify a distal fluid flow and reduce a lung volume, as described herein. Each of these valves include a retrieval section 716 and an engagement feature 730 coupled to a proximal end of the valve 700. The engagement feature 730 is disposed proximally and aligned with a central longitudinal axis 70. The valves 700 further include the retrieval section 716 having two or more struts 718 extending from the proximal end 726 to an outer edge of the valve. As the engagement feature is withdrawn proximally, optionally into a delivery catheter 90, the struts 718 urge the outer edges of the valve radially inwards disengaging the anchors 720 from the wall prior to proximal movement and mitigating damage to the walls of the target location. As described herein, the retrieval section 716 can be formed integrally with the valve 700 or can be coupled to the valve 700 using adhesive, bonding, welding, ultrasonic welding, RF welding, or the like. Advantageously, the retrieval section 716 can be coupled to existing endobronchial valves to facilitate retrieval or repositioning of the endobronchial valve. Alternatively, or in addition, the swellable material coating can be applied to the existing endobronchial valve to facilitate the gradual reduction in lung volume and mitigate associated complications.

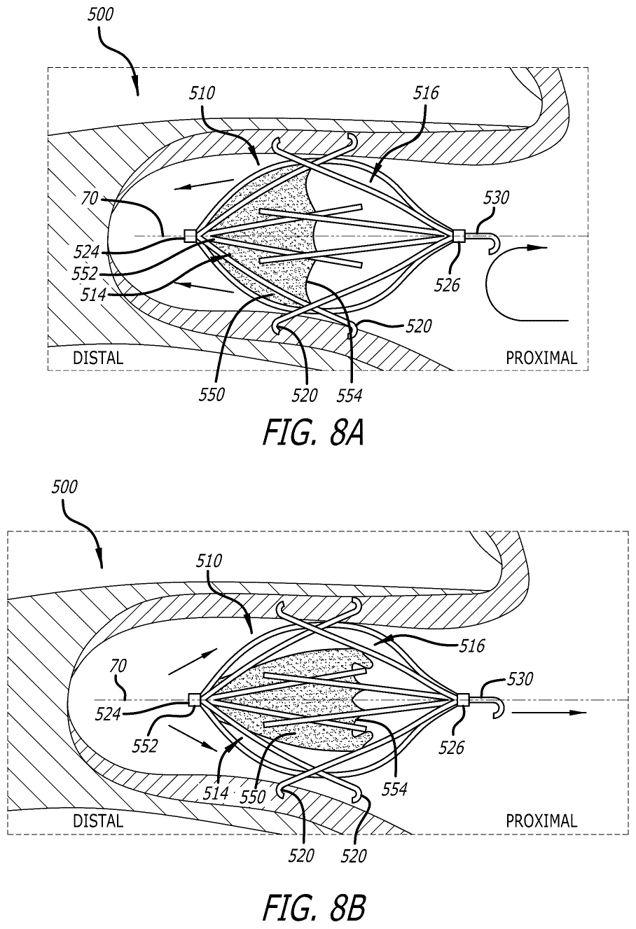

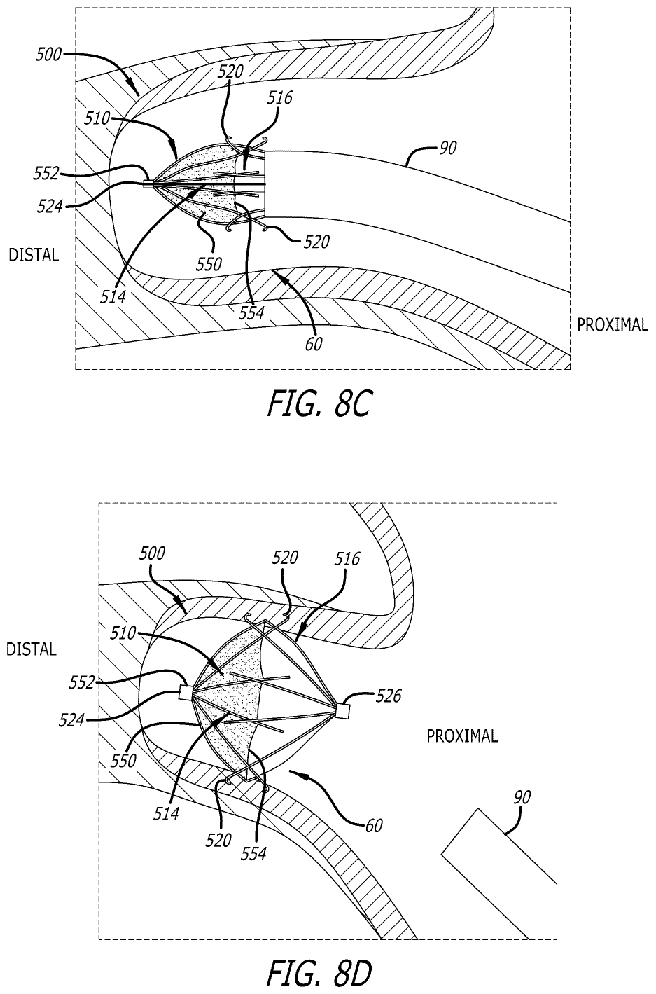

FIGS. 8A-8D show an embodiment of an endobronchial valve 500 generally including a frame 510 and a valve member 550. The frame 510 includes a first plurality of arms 514 coupled to a distal end 524 of the frame 510 and extending proximally. The proximal end of each arm of the first plurality of arms 514 includes an anchor 520 configured to engage a wall of the target location and mitigate one or both of proximal and distal migration. The frame 510 further includes a second plurality of arms 516 coupled to a proximal end 526 of the frame 510 and extending distally. The distal end of each arm of the second plurality of arms 516 includes an anchor 520 configured to engage a wall of the target location and mitigate one or both of distal and proximal migration. In an embodiment, an arm of the first plurality of arms 514 can hingedly engage an arm of the second plurality of arms 516 to secure the first plurality of arms 514 to the second plurality of arms 516.

In an embodiment, the valve 500 further includes a valve member 550 defining a cone shape, or an “umbrella” shape, and formed of a flexible membrane material including a plastic, polymer, elastomer, rubber, silicone rubber, polytetrafluoroethylene (PTFE), expanded polytetrafluoroethylene (ePTFE), polyurethane (PU), gaseous impermeable material, or similar suitable material. An apex 552 of the valve member 550 is coupled to the distal end 524 of the frame 510. Further, a portion of the base 554 of the valve member can be coupled an arm of the first plurality of arms 514.

As shown in FIG. 8A, distal fluid flow can impinge on an inner surface of the valve member 550 and fill the cavity defined by the valve member 550. The valve member 550 can expand radially outwards, relative to a central longitudinal axis 70, and mitigate or prevent distal fluid flow past the valve 500. As shown in FIG. 8B, a proximal fluid flow can impinge on an outer surface of the valve member 550 and urge the valve member 550 radially inwards, relative to a central longitudinal axis 70, to allow proximal fluid flow with little to no resistance.

In an exemplary method of use, as shown in FIGS. 8C-8D, an endobronchial valve 500 is provided as described herein. The valve 500 can be transitioned to a collapsed configuration as shown in FIG. 8C. The proximal ends of the first plurality of arms 514 are deflected radially inwards, the valve member 550 is folded such that the base 554 is deflected radially inwards and the distal ends of the second plurality of arms 516 are deflected radially inwards. In an embodiment, the valve 500 is biased to the expanded configuration and retained in the collapsed configuration by the delivery catheter 90. The valve 500 and delivery catheter 90 assembly is then advanced to the target location within a bronchus.

The valve 500 can be advanced from the delivery catheter 90, optionally by urging the valve 500 distally using a stylet 80. The first plurality of arms 514 and the second plurality of arms 516 then transition to the expanded configurations and engage a wall of the target location (FIG. 8D). As the first plurality of arms 514 expand, the portions of the base 554 that are coupled to the arms transitions the valve member 550 to an expanded configuration.

During inhalation, distal fluid flow can impinge on an inner surface of the valve member 550 causing the valve member 550 to expand and reduced distal fluid flow. Optionally, the base 554 can impinge on a wall of the target location and prevent distal fluid flow. In the alternative, some fluid flow can pass between the base 554 and the wall of the target location to provide a reduced distal fluid flow. During exhalation, proximal fluid flow can impinge on an outer surface of the valve member 550 and urge the valve member 550 radially inward providing relatively little or no resistance to proximal fluid flow.

In an embodiment, the valve 500 can be retrieved or repositioned by advancing the delivery catheter 90 to the target location, advancing a stylet 80 through the delivery catheter lumen 92, engaging the distal tip of the stylet 80 with an engagement feature 530 coupled with the proximal end 526 of the frame 510 and withdrawing the stylet 80 proximally. By withdrawing the stylet 80 proximally, the second plurality of arms 516 are deflected radially inwards disengaging the distal ends of the second plurality of arms 516 from the wall of the target location. Further, the second plurality of arms 516 are hingedly engaged with the first plurality of arms 514. As such, as the second plurality of arms 516 are deflected radially inwards the first plurality of arms 514 are also deflected radially inwards, also disengaging the proximal ends from the wall of the target location. As the first plurality of arms 514 are deflected radially inwards, the valve member 550 coupled thereto is folded to the collapsed configuration. Once in the collapsed configuration the valve 500 can be withdrawn into the delivery catheter lumen 92 for removal or repositioning.

Advantageously, the engagement feature and the reversible transitioning of the valves between the collapsed and expanded configurations allow the valves disclosed herein to be easily retrieved and/or repositioned after the initial placement, without having to invert the device, or stretch or otherwise cause damage to the target location.

Advantageously, allowing some distal fluid flow on inhalation, and therefore reducing subsequent proximal fluid flow on exhalation, allows for the gradual removal of secretions from the portion of the lung that is being isolated. Further, by modifying the rate of distal fluid flow over time allows for a gradual reduction in lung volume, mitigating the complications associated with a sudden reduction in lung volume. For example, a series of valves can be placed, retrieved and replaced with a second or subsequent valves, each designed to have a progressively reduced level of distal fluid flow so as to allow healthy portions of the lung to adapt to the change in lung volume over time, mitigating complications such as pneumothorax.

Alternatively, or in addition to, the rate of distal fluid flow for a valve can change progressively over time. As such, once placed, the rate of proximal fluid flow gradually decreases, mitigating complications from sudden lung volume changes. For example, one or more valves described herein can include a swellable material configured to swell in size over a predetermined period of time and gradually reduce the distal fluid flow through the valve. For example, a swellable material, hydrogel, or similar suitable material can coat a lumen of the frame, a slit opening, and/or a valve member such as those described herein. Once placed, the swellable material can gradually absorb moisture from the surrounding air flow and start to expand, reduced the cross-sectional area for distal fluid flow and gradually modifying lung volume over time.

In embodiments, the valve frames disclosed herein are elastically deformable and biased towards the expanded configuration. Alternatively, or in addition, the valve frames are malleable and transitionable to the expanded configuration by balloon expansion, or similar mechanical means. In an embodiment, the valve frames disclosed herein are formed of a shape memory material configured to transition between an expanded configuration and a collapsed configuration in response to a trigger, for example a change in temperature trigger. However, other triggers are also contemplated. These and other means of transitioning the frames to the expanded configuration are considered to fall within the scope of the present invention.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A valve for placement in an airway to modify lung volume, comprising:
a frame having a body extending along a longitudinal axis between a proximal end and a distal end, the frame transitionable between a collapsed configuration and an expanded configuration; and
a valve member formed of a flexible material and coupled to the frame, comprising:
a distal valve member having a conical shape extending between an apex disposed distally and a base disposed proximally and defining a cavity communicating with an opening at the base, the base defining a first diameter; and
a proximal valve member defining a frusto-conical shape extending between a distal end and a base, the distal end having an opening and defining a second diameter (D2) less than the first diameter (D1), the base having an opening and defining a third diameter (D3) larger than the first diameter (D1), the base of the distal valve member disposed through the distal end opening of the proximal valve member.

2. The valve according to claim 1, wherein one or both of the base of the distal valve member and the base of the proximal valve member is configured to deflect radially inwards to provide proximal fluid flow, and where the base of the distal valve member impinges on the distal end opening of the proximal valve member to provide a reduced fluid flow.

3. The valve according to claim 2, wherein the base of the distal valve member includes a plurality of ridges and troughs disposed about a circumference of the base and configured to provide one or more gaps for distal fluid flow when the distal valve member base engages the proximal valve member distal opening.

4. The valve according to claim 3, wherein one or both of the distal valve member and the proximal valve member includes a coating having a swellable material configured to expand over a predetermined time frame and occlude the one or more gaps to reduce or prevent distal fluid flow.

5. The valve according to claim 1, wherein one or both of the distal valve member and the proximal valve member includes a plurality of apertures to allow fluid flow therethrough in both the proximal direction and the distal direction.

6. The valve according to claim 5, wherein one or both of the distal valve member and the proximal valve member includes a coating having a swellable material configured to expand over a predetermined time frame and occlude the plurality of apertures to reduce or prevent distal fluid flow.

7. The valve according to claim 1, wherein the frame body defines cylindrical shape having a diameter in the expanded configuration that is equal to, or larger than a diameter of a target location, the frame body includes one or more anchors that protrude from the frame body in the expanded configuration to engage a wall of the target location to prevent migration of the valve.

8. The valve according to claim 1, wherein the frame body includes a lattice structure of a plurality of struts, the frame further includes a distal section including two or more struts extending from the frame body to the distal end of the frame to form a conical shape and configured to retain the valve therein.

9. The valve according to claim 1, wherein the frame further includes a proximal section including two or more struts extending from the frame body to the proximal end of the frame, the proximal end includes an engagement feature.

10. The valve according to claim 1, further including one or more flexible members coupling a perimeter of the base of the proximal valve member to the frame and configured to allow the proximal valve member to move along the longitudinal axis relative to the frame between an open and a closed position.

11. The valve according to claim 1, further including a biasing member coupling the distal end of the proximal valve member to the distal end of the frame and configured to allow the proximal valve member to move along the longitudinal axis relative to the frame between an open and a closed position.

12. The valve according to claim 1, wherein a perimeter of the base of the proximal valve member is coupled to the proximal end of the frame body and further includes a proximal section coupled to the proximal end of the frame body, the proximal section including a plurality of struts extending from the frame body to a proximal end of the frame, the proximal end including an engagement feature.

13. The valve according to claim 1, wherein one or both of the distal valve member and the proximal valve member includes a relatively rigid section disposed distally and a relatively flexible section disposed proximally.

14. A method of placing a valve at a target location for modifying a lung volume, comprising:
advancing the valve in a collapsed configuration and disposed within a delivery catheter to the target location, the valve including a frame and a valve member;
transitioning the valve to an expanded configuration for an anchor of the frame engages a wall of the target location; and
transitioning the valve member to an expanded configuration to reduce distal fluid flow across the valve.

15. The method according to claim 14, further comprising:
coupling a distal end of a stylet, disposed within a catheter lumen, with an engagement feature at a proximal end of the frame;
withdrawing the stylet proximally into the catheter lumen;
impinging a strut of a proximal section of the frame on an entrance of the catheter lumen;
levering the frame radially inwards to disengage the anchor away from the wall of the target location;
transitioning the frame to the collapsed configuration; and
withdrawing the valve into catheter lumen.

16. The method according to claim 14, wherein the valve member transitions between an open position to allow proximal fluid flow, and a closed position to modify distal airflow, an outer surface of a base of a distal valve member impinges on an inner surface of a distal opening of a proximal valve member.

17. The method according to claim 16, wherein the valve member in the open position further includes deflecting the base of the distal valve member radially inward to provide a gap between the base of the distal valve member and the opening of the proximal valve member.

18. The method according to claim 16, wherein the valve member in the open position includes sliding the proximal valve member relative to the frame, the valve further including a flexible member coupled to a base of the proximal valve member, or a biasing member coupled to an apex of the valve, biasing the proximal valve member to the closed position.

19. A valve system for modifying a lung volume, comprising:
a valve member defining a conical shape extending proximally from an apex to a base, and defining a cavity communicating with an opening at the base;
a frame including a plurality of struts extending distally from a proximal end of the frame to the base of the valve member, and two or more arms extending from the proximal end of the frame to engage a wall of a target location; and
an engagement feature coupled to the proximal end of the frame.

20. The valve system according to claim 19, wherein the arm further includes one or more anchors configured to engage the wall and mitigate movement of the valve system in one or both of a proximal direction and a distal direction.

21. The valve system according to claim 20, further including a delivery catheter and a stylet disposed within a lumen of the delivery catheter, the stylet configured to engage the engagement feature and withdraw frame into the catheter lumen, the two or more arms configured to engage the catheter to lever the anchors radially inwards away from the wall of the target location.
